(19) United States
(12) Patent Application Publication
(10) Pub. No.: US 2011/0207207 A1
(43) Pub. Date: Aug. 25, 2011

(54) MICROFLUIDIC CELL SORTER UTILIZING BROADBAND COHERENT ANTI-STOKES RAMAN SCATTERING

(76) Inventors: Emily A. Gibson, Boulder, CO (US); Tim C. Lei, Thornton, CO (US)

(21) Appl. No.: 13/126,412

(22) PCT Filed: Oct. 28, 2009
(86) PCT No.: PCT/US2009/062419

§ 371 Date: Apr. 27, 2011

Related U.S. Application Data
(60) Provisional application No. 61/197,548, filed on Oct. 28, 2008.

(51) Int. Cl. C12M 1/34

(52) U.S. Cl. 435/288.3; 435/288.7

(57) ABSTRACT

A microfluidic cell sorter has a microfluidic structure (500) with a sample input channel (1 1 0) leading into an observation region (214), two buffer channels (1 1 8) configured to hydrodynamically focus a sample target cell (208) within the observation region, and at least two output channels (1 1 4). Apparatus directs the target cell into a selected output channel based on a cell sorting control signal (61 6). A CARS pulse source (524) generates CARS pulses (526), which are directed to the target cell within the observation region. A detector (530) detects CARS illumination scattered from the target signal and generates a spectrum signal based on the detected illumination. A processor (61 4) identifies the target cell based on the spectrum signal and generates the cell sorting control signal based on the identity of the target cell.

### BACKGROUND OF THE INVENTION

1. Field of the Invention

The present invention relates to apparatus for measuring and sorting cells within a microfluidic structure using broadband coherent anti-Stokes Raman scattering (CARS).

2. Description of the Related Art

The general idea of flow cytometry is to perform rapid measurements on cells flowing in a hydro-dynamically focused single stream. These measurements are typically optical (i.e. light scattering, fluorescence) because of the inherent sensitivity and fast data rates. The data rate determines the number of cells that can be measured in a given time. In current cytometers, typical rates of ˜5,000 to 10,000 cells/s are used to obtain good statistics. The disadvantages of flow cytometry are its size and non-portability, the requirement for a sterile lab environment, the need for large sample volumes, and its limited chemical analysis capabilities.

Raman spectroscopy measures the vibrational spectrum of biomolecules and can distinguish between different cell types, viruses, and other pathogens. In Raman scattering, the fundamental laser (pump) is scattered by a vibrational mode, producing a lower energy (Stokes) or higher energy (anti-Stokes) photon.

The Raman spectrum provides a measure of the vibrational mode density that can be translated to biochemical content. Traditional Raman spectra of a variety of biomolecules and cells have been measured, showing the capability of this technique to identify different cells types, bacteria, and viruses. Raman spectroscopy can also distinguish between healthy and unhealthy cells, and cancerous and non-malignant cells. Thus, Raman spectroscopy is a powerful technique for label-free identification and characterization. Unfortunately, despite these advantages, this technique has limited use for biomedical applications due to the long acquisition times required for the measurement. In the Raman process, a narrow band laser illuminates the sample and a portion of the incident photons are scattered by interactions with molecular vibrations, resulting in a shift to higher (anti-Stokes) or lower frequency (Stokes) photons. The signal intensity is very weak because of the extremely low scattering cross-section (˜10⁻³⁰ cm²/molecule).

CARS is a nonlinear optical process that selectively and coherently excites vibrational resonances of biomolecules to rapidly obtain the Raman (vibrational) spectrum. Compared to traditional Raman scattering, the CARS process increases the detection sensitivity by 10⁷ to allow rapid data acquisition. With the associated decrease in measurement times, CARS has been applied in biomedical microscopy to image live cells at video rates without extrinsic fluorescence dye labeling. Two photons (pump and Stokes) excite a specific vibrational resonance coherently. A third photon (probe) subsequently measures the density of the vibrational resonance. The density of the vibrational resonance is proportional to number of emitted anti-Stokes photons that are energy shifted by that vibrational mode. A traditional CARS setup uses two synchronized picosecond lasers or a single picosecond laser with an optical parametric amplifier to generate the two laser beams with the difference in frequencies matched to one particular vibrational resonance. The disadvantage of this approach is its difficulty in frequency tuning needed to measure a full spectrum (as opposed to a single vibrational mode). Mechanical movement or temperature control, such as rotation or heating of an optical crystal, is needed to tune the optical frequencies of the two lasers, resulting in misalignment or very slow frequency tuning rate of the system. Therefore, this two-laser approach is not suitable for flow cytometry and sorting because measuring a single Raman resonance is not adequate to separate a wide range of cell types. Instead a full spectrum inside the Raman fingerprint region (300 cm⁻¹ to 1800 cm⁻¹) or several different Raman signature frequencies are needed in order to characterize the chemical composition of a cell. Another advantage of acquiring the full spectrum is that by taking the ratio of different spectral regions, it is possible to identify different cell types without calibration because a ratiometric measurement is not dependent upon the overall magnitude of the signal.

In contrast to CARS with two narrow linewidth lasers, in broadband CARS, a broadband Stokes beam excites many modes simultaneously, which allows the entire vibrational spectrum to be measured in a single shot. In broadband CARS, since the broadband laser beam is used to excite many modes, a higher laser intensity is needed to measure the output signal resulting in a non-resonance background. Fortunately, this background can be eliminated and the Raman spectrum retrieved using a variety of phase and polarization pulse shaping methods.

### SUMMARY

A micro-fluidic cell sorter utilizes a coherent broadband laser to implement broadband CARS flow cytometry. A preferred embodiment combines multiplex Coherent Anti-Stokes Raman Scattering (CARS) spectroscopy using a broadband coherent laser source with a microfluidic device with hydrodynamic focused channel for label free cell characterization, quantification, and sorting.

A microfluidic cell sorter has a microfluidic structure with a sample input channel leading into an observation region, two buffer channels configured to hydrodynamically focus a sample target cell within the observation region, and at least two output channels. Apparatus directs the target cell into a selected output channel based on a cell sorting control signal. A CARS pulse source generates CARS sources, which are directed to the target cell within the observation region. A detector detects CARS illumination scattered from the target signal and generates a spectrum signal based on the detected illumination. A processor identifies the target cell based on the illumination signal and generates the cell sorting control signal based on the identity of the target cell.

In one embodiment, back-pressure is applied to the non-selected output channels so that the target cell exits the selected output channel. The microfluidic structure might comprise a silicon structure anodically bonded between two pyrex slides.

The CARS source might comprise a broadband laser source which provides the pump, probe and Stokes frequencies as well as a pulse shaper. The broadband laser source could be a femtosecond laser and a photonic crystal fiber to broaden the optical spectrum of the pulse. The photonic crystal can also amplify the intensity of the probe frequency. Or, the broadband laser source could comprise a fiber laser.

In one embodiment, the pulse shaper delays the probe wavelength to improve the CARS output signal.

Similarly, a microfluidic flow cytometer for examining cells in an input sample includes a microfluidic structure including a sample input channel leading into an observation region and two buffer input channels configured to hydrodynamically focus a continuous stream of cells in a single file line within the observation region. A coherent anti-Stokes Raman scattering (CARS) pulse source generates CARS pulses, and apparatus for detecting the presence of each of a series of target cells as they enter the observation region triggers the CARS pulse source accordingly. The CARS pulses are directed into the target cells within the observation region. Then, a detector detects CARS illumination scattered from each target cell and generates a spectrum signal based upon the detected illumination. A processor analyzes each target cell based upon its associated spectrum signal.

The present invention can measure the spectrum of a cell in around 1-10 ms, resulting in a data rate on the order of 100-1000 cells/second, fast enough for flow cytometry applications in real time.

### BRIEF DESCRIPTION OF THE DRAWINGS

FIG. 1 is a schematic diagram illustrating a microfluidic cell sorter according to the present invention.

FIG. 2 is a schematic diagram illustrating a second microfluidic cell sorter according to the present invention.

FIGS. 3A-3D are side cut-away views showing the process of fabricating microfluidic cell sorters according to the present invention.

FIG. 4 is a flow diagram showing a process of data acquisition using a micro-fluidic cell sorter according to the present invention.

FIG. 5A is a plot of a photodiode signal detected in the system of FIG. 4. FIG. 5B is a schematic diagram showing a data gathering configuration in the system of FIG. 4.

FIG. 6 is block diagram showing apparatus for measuring and sorting cells within a microfluidic structure using broadband CARS.

FIGS. 7A-7D are plots illustrating the formation of the CARS signal of FIG. 6.

FIG. 8 is a block diagram illustrating an embodiment of a system for measuring and sorting cells within a microfluidic structure using broadband CARS.

### DETAILED DESCRIPTION OF THE PREFERRED EMBODIMENTS

The following reference numbers are used in the figures:

| | |
|---|---|
| 101 | Microfluidic channels |
| 102 | Target output |
| 104 | Waste output |
| 106, 108 | Buffer reservoirs |
| 110 | Sample reservoir |
| 112 | Focusing junction |
| 114 | Output channels |
| 116 | Input channel |
| 118 | Buffer channels |
| 202 | Sample fluid |
| 204 | Sample cells |
| 206 | Buffer fluid |
| 208 | Target cell |
| 210 | Light scattering measurement location |
| 212 | Broadband CARS measurement location |
| 214 | Observation region |
| 302 | UV light |
| 304 | Mask |
| 306 | Silicon |
| 308 | Photoresist |
| 310 | Glass slide |
| 500 | Microfluidic flow cytometer |
| 502 | Time at focusing junction |
| 504 | Time at light scatter detect |
| 506 | CARS acquisition time |
| 510 | Time of cell sorting |
| 512 | Light source |
| 514 | Light at Measurement point 210 |
| 516 | Light scattered from cell |
| 520 | Photodetector |
| 524 | CARS input pulse source |
| 526 | CARS input pulse |
| 528 | Scattered CARS radiation |
| 530 | Light detector |
| 532 | Line detectors |
| 536 | Back pressure |
| 602 | Laser |
| 604 | Pulse spreader |
| 606 | Pulse shaper |
| 612 | Filter |
| 614 | Processor |
| 616 | Sort control signal |
| 618 | Display/store data element |
| 802, 816 | Optical Gratings |
| 804, 814 | Lenses |
| 806 | Spatial Light Modulator (SLM) |
| 808 | Spatial filter |
| 810 | Optical delay unit |
| 812 | Mirrors |
| 818 | Galvo mirror |
| 822 | Pump beam and Stokes beam |
| 824 | Probe beam |

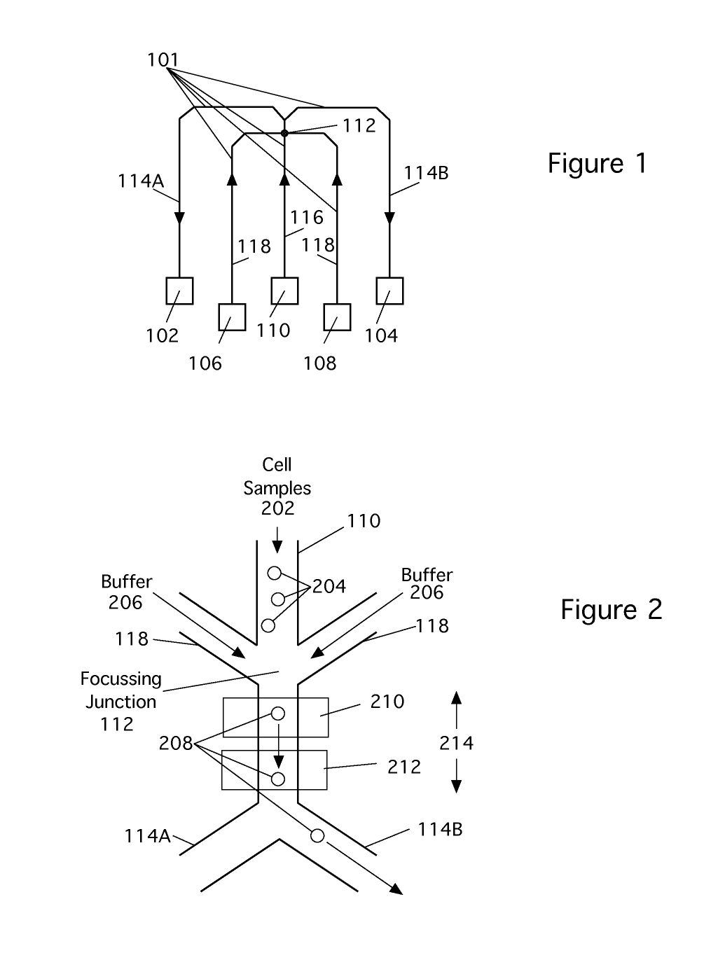

FIG. 1 is a schematic diagram illustrating a first embodiment of a microfluidic cell sorter according to the present invention. FIG. 2 is a simplified plan view of the microfluidic device of FIG. 1.

The microfluidic device of FIGS. 1 and 2 consists of three input channels, a junction 112 where the sample is hydrodynamically focused by buffer solution, an observation region that is substantially optically transparent in the wavelength range used for excitation and detection, and two exit channels.

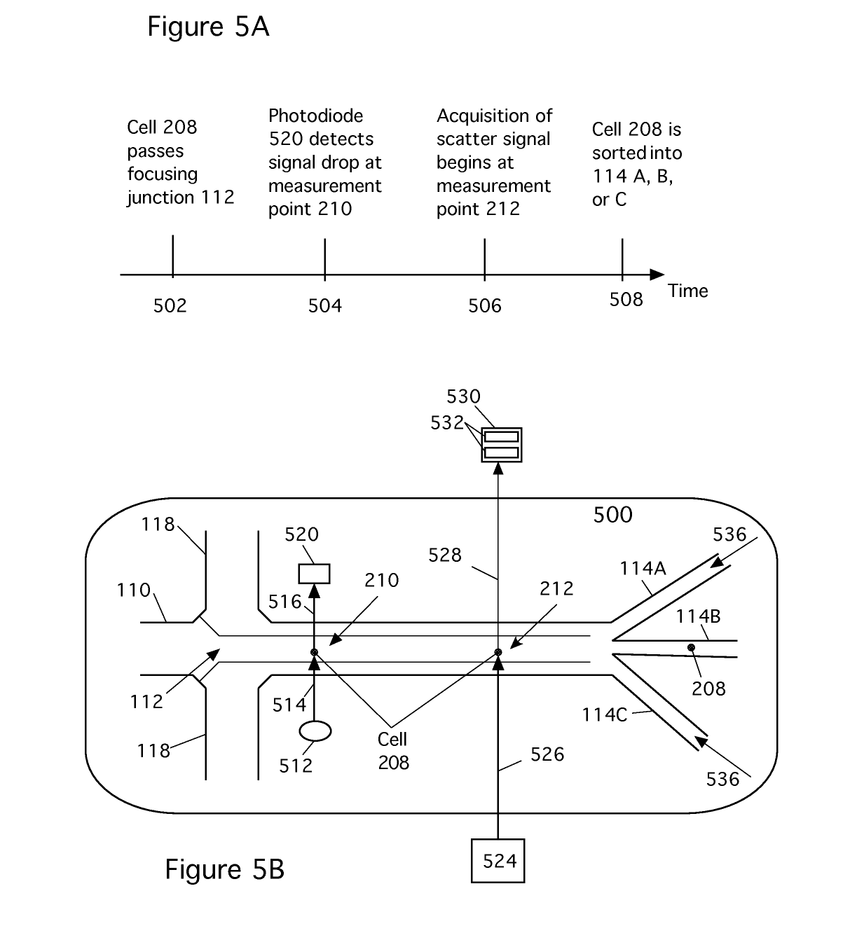

Sample reservoir 110 provides sample fluid 202 containing cells 204 to the cell sorter via input channel 116. Buffer reservoirs 106, 108 provide buffer 206 (via two buffer channels 118) to the microfluidic device for the purpose of hydro-dynamically focusing the sample stream 202 at focusing junction 112. This permits only one target cell 208 at a time to pass through the observation region 214. Observation region 214 includes a light scattering measurement location 210, where cell 208 is detected in order to trigger CARS measurement, and CARS measurement location 212, where the scattered output CARS illumination from the sample is detected. After cell 208 passes through observation region 208 it is sorted into target output reservoir 102 or waste output 104 via output channels 114 A and B. FIG. 5B shows a sorting process and the illumination and detection apparatus. As an example, the fluidic resistance of the two exit channels is adjusted so that the focused cell sample flows into the waste output channel 114B by default. When a cell 208 of interest is detected, back-pressure is applied to the waste channel using a fast pressure controller to quickly direct the focused streamline into the target output channel 114A. FIG. 5B also shows the hydrodynamic focusing of the sample by the buffer solution. The focusing is necessary in order to direct the cells single file in the center of the channel for optical measurement and in order to control their direction in the outlet sort or waste channels. At the first measurement point 210, the cell is detected by light scattering on a photodiode. The signal from the photodiode triggers the CARS acquisition at the second measurement point 212.

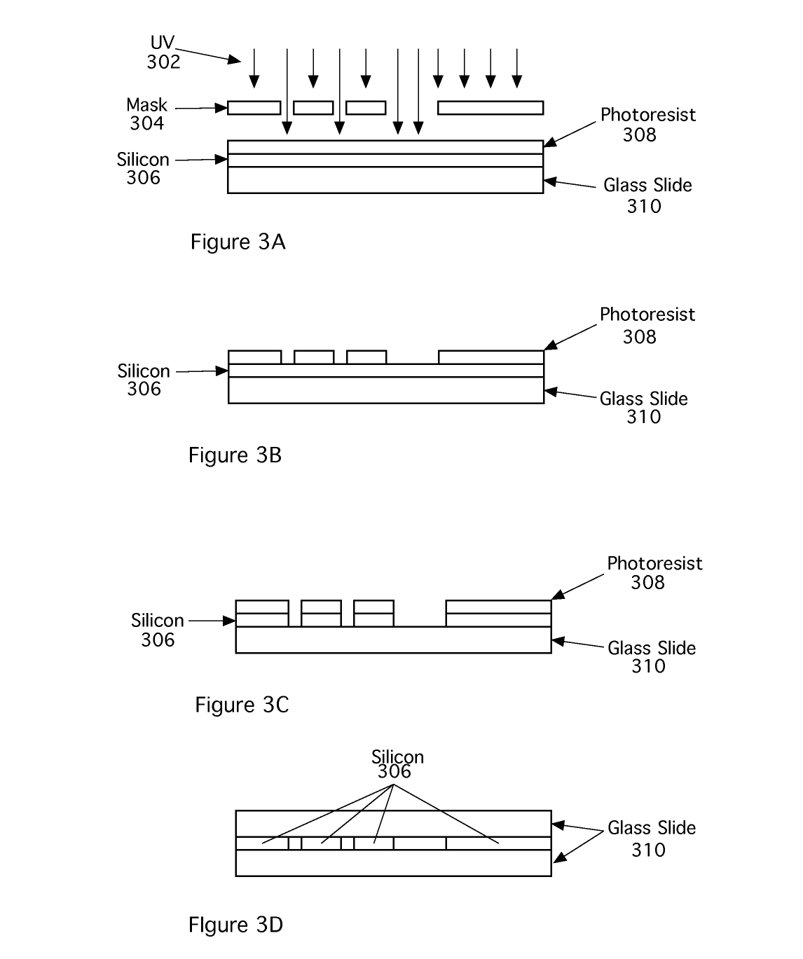

In a preferred embodiment, the channel widths range from 100-400 microns. The channel heights are 25-30 microns. FIGS. 3A-D show an example of a process of forming the microfluidic channels. The on-chip reservoirs are made of PEEK biocompatible plastic. The sample and buffer solutions are loaded into reservoirs on the microfluidic device. The reservoirs hold approximately 250 microliters. They are sealed to the inputs of the microfluidic channels by o-rings pressed onto the device by a clamp and two screws. The tops of the reservoirs have threaded connectors to pressure tubing where pressure can be applied to create flow of the input and buffer solution in the microfluidic input channels and for pressure switching at the exit channels. The waste and target output are collected in the reservoirs 102, 104 at the exit channel connections.

Alternative methods for creating fluid flow in microfluidic devices include electroosmotic flow and integrated miniature fluidic pumps.

FIGS. 3A-3D are side cut-away views showing one process of fabricating microfluidic cell sorters according to the present invention. The microfluidic device is constructed as follows. A double sided polished pyrex substrate 310 is anodically bonded to a double sided polished silicon wafer 306. The wafer is lapped and polished to a thickness of 25 microns. The silicon wafer 306 attached to the pyrex 310 is coated with SU-8 photoresist 308. Mask 304 is placed over the photoresist layer 308. Mask 304 is open in the area where the microfluidic channels will be created. Using UV lithography and reactive ion etching, the microfluidic channels are created in the silicon wafer as shown in FIGS. 3A-3D.

FIG. 3A shows the UV illumination 302 incident on photoresist layer 308 everywhere except where it is blocked by mask 304. FIG. 3B shows that the photoresist has been removed at the location of the channel features by UV lithography 302. There is a layer of photoresist SU-8 everywhere else.

Next, the substrate is placed in the reactive ion etch and the channels are etched into the silicon wafer completely removing all the silicon down to the pyrex substrate, as shown in FIG. 3C. In FIG. 3D, the remaining SU-8 is removed from the silicon, and a second glass slide 310 is adhered to the top of silicon layer 306. In this embodiment, a second pyrex substrate is drilled with holes at the locations of the start of the input and exit channels (see FIGS. 1, 2, and 5B). The second pyrex piece is anodically bonded to the silicon/pyrex substrate forming pyrex/silicon/pyrex layers and sealing the microfluidic channels. The holes are used to input and extract solutions from the microfluidic channels. The use of two glass slides means the microfluidic device is transmissive.

Alternative methods for manufacturing microfluidic devices with optical transparency include: reactive ion etching in glass or laser machining in glass and glass/glass bonding. Use of SU-8 photoresist as the layer containing the channels may replace the silicon wafer and adhered to the glass with epoxy.

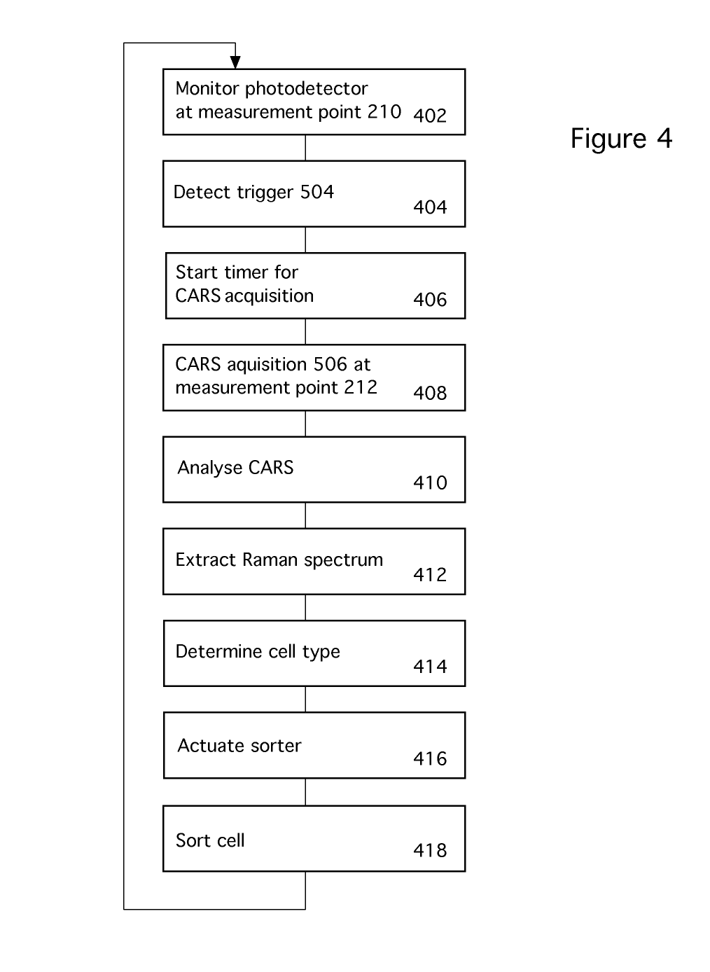

FIG. 4 is a flow diagram showing a process of data acquisition using a micro-fluidic cell sorter and broadband CARS analysis according to the present invention. Refer also to FIGS. 5A and 5B. In the embodiment of FIG. 4, photodetector 520 monitors light from light source 512 at measurement point 210. When cell 208 reaches measurement point 210, light 514 is scattered by cell 208, so the amount of light 516 detected by detector 520 decreases. Step 404 indicates detection of this decrease. In step 406, the detection triggers a timer. When the timer elapses, cell 208 reaches broadband CARS measurement location 212, and CARS acquisition occurs in step 408. In step 410, the CARS data is analyzed, and in step 412, the Raman spectrum is extracted. Cell 208 is identified in step 414. Step 416 actuates the sorter, and cell 418 is sorted into its output channel in step 418.

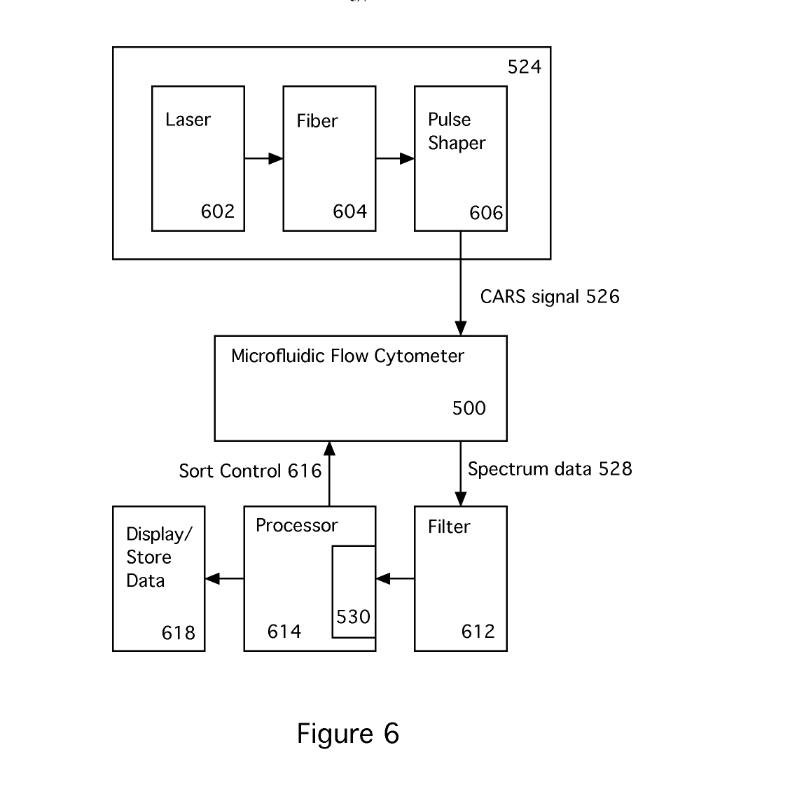

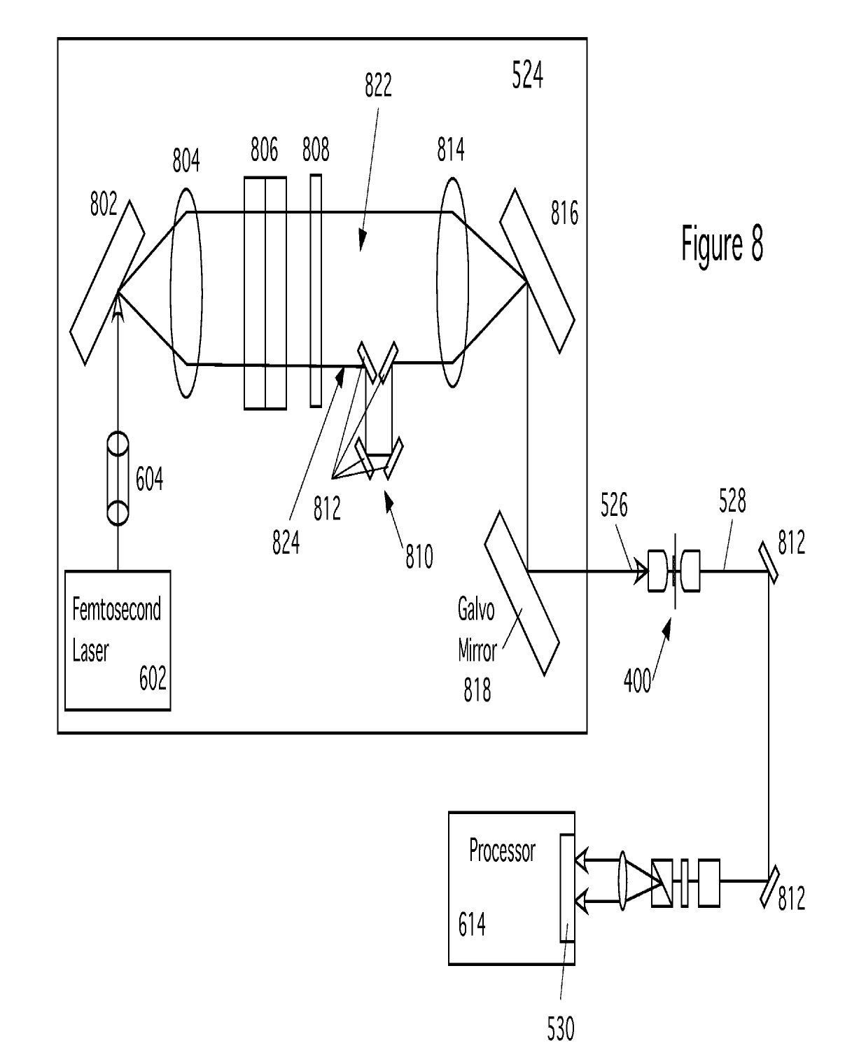

FIG. 5A is a timeline of events within microfluidic device 500 of FIG. 5B. Device 500 is similar to those shown in FIGS. 1 and 2, except that it includes three output channels 114. FIG. 5 also shows more details and includes some of the apparatus for CARS analysis. At time 502, cell 208 passes through focusing junction 112. Buffer 206 enters device 500 via buffer channels 118, and moves cell 208 to the center of the flow as it passes through focusing junction 112 and enters observation region 214. At time 504, cell 208 reaches measurement point 210, and detector 520 detects a signal drop. At time 506, cell 208 reaches measurement point 212 and acquisition of the CARS scatter signal occurs. CARS input pulse source 524 is shown in more detail in FIGS. 6 and 8. Detector 530 detects the scattered CARS. In this embodiment, detector 530 comprises two line detectors 532, for example single line CCD chips. This eliminates the bottle neck of a 2-dimensional CCD camera.

At time 508, cell 208 is sorted into channel 114B. In this embodiment, the sorting is accomplished by creating back pressure 536 at the other two exit channels 114A and 114C.

Alternative methods for switching cells to the target exit channel(s) include electro-osmotic switching by switching an applied voltage to the target exit channel, switching using fluidic pumps, optical trapping by laser fields, and dielectric trapping by integrated electrodes with applied voltages.

FIG. 6 is a high-level block diagram showing apparatus for measuring and sorting cells within a microfluidic structure using broadband CARS. As an example, CARS input pulse source 524 includes a laser 602 for generating broadband pulses, a fiber 604 for spreading the pulses, and a pulse shaper 606 for configuring the pulses to accomplish broadband CARS. CARS input pulses 526 are provided to micro-fluidic flow cytometer 500 as shown in FIG. 5B. The scattered CARS spectrum is output to a filter 612, and then to a processor 614 including detector 530. Processor 614 controls the cell sorting procedure via signal 616. It also provides data on the detected cell to storage or display 618.

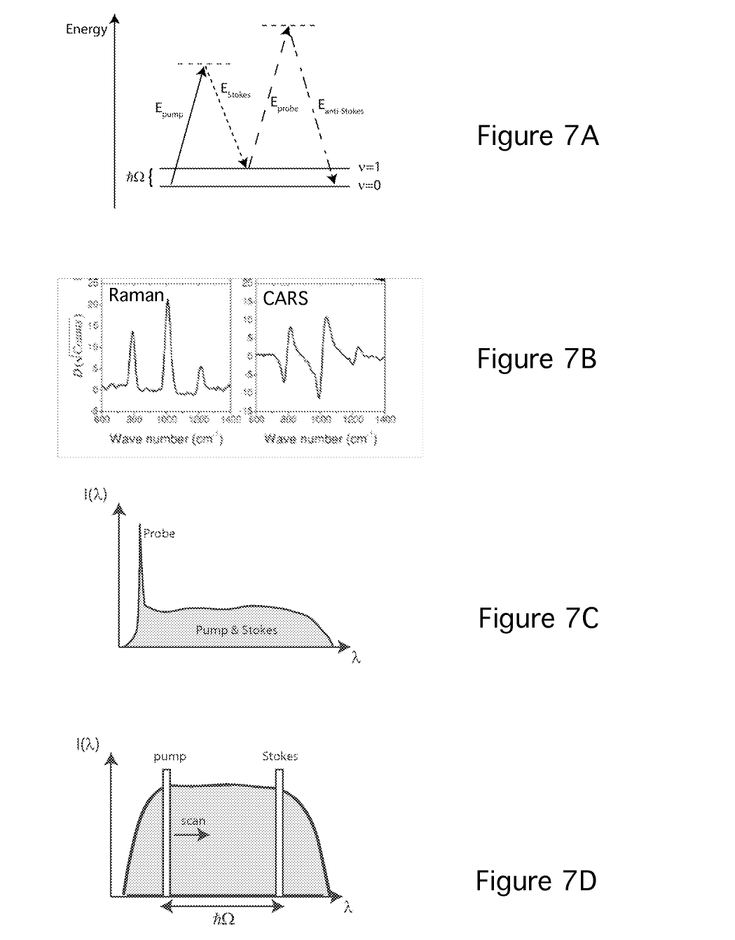

FIGS. 7A-7D are plots illustrating the process of configuring of the CARS signal 526 of FIG. 6. FIG. 7A shows an energy schematic diagram of the basic CARS process. Here, three different optical laser frequencies (pump, Stokes and probe) are sent into the sample to initiate the CARS process. An anti-Stokes photon will result from the process and emit, with the highest photon energy well separated from the rest of the frequencies, for detection. The intensity of the anti-Stokes emission is proportional to the number of vibrational oscillators in the sample. A CARS spectrum can be built up through scanning the vibrational frequencies by using different pump and Stokes frequencies to match the vibrational frequencies.

FIG. 7C shows the process of tailoring the optical spectrum to enhance the intensities of the probe wavelengths. The higher intensity probe allows more efficient generation of the CARS signals even if the number of vibrational oscillators in the sample is relatively low.

FIG. 7B shows that the CARS spectrum (on the right) is different from the spontaneous Raman spectrum (on the left) acquired by a continuous wave (CW) laser source. The difference arises from the fact that the instantaneous four-wave mixing process or the so-called “non-resonance background” can be generated even though the vibrational resonance (ₕΩ) is absent. This non-resonance background signal coherently mixes with the coherent vibrational signal and results in the CARS spectrum rather than the conventional Raman spectrum. The disadvantages of the CARS spectrum mixing with the non-resonance background are 1) the non-resonance background can be so large that it overwhelms the vibrational signatures and makes cell identification difficult; and 2) the CARS spectrum is significantly different from the spontaneous Raman spectrum, which makes direct comparisons between the CARS spectrum and the Raman spectrum difficult.

There are several approaches to solve this problem. FIG. 7D shows a first approach, based on methods by S. Lim et al (Physical Review A 72, 41803), which use polarization and phase modulation of the probe frequencies to extract the Raman spectrum. The approach requires applying a 90 degree (π/2) phase shift to the probe frequency, in addition to generating the CARS signals in two different polarizations. The CARS signals generated in these two perpendicular polarizations are subsequently mixed with an optical setup composed of a Fresnel rhomb and a Wollaston prism. After mixing the signals with the optical setup, the resulting signals, having two different polarizations, are then sent to a two-dimensional spectrometer for signal recovery. By subtracting the two spectra acquired in the two different polarizations through the 2D spectrometer, the Raman spectrum can be completely recovered and the non-resonance background signal can be removed. Although this approach is robust, it is complex and isn't necessarily required for CARS cell sorting, especially if the CARS signal is strong enough for direct comparison.

FIG. 8 is a block diagram illustrating a preferred embodiment of a system for measuring and sorting cells within a microfluidic structure 500 using broadband CARS. Laser 602 comprises a femtosecond laser. For example, a Titanium doped Sapphire (Ti:Sa) femtosecond laser can be used as the light source. Picosecond, nanosecond and broadband continuous-wave (cw) lasers can also be used as long as phase coherency is maintained across the spectrum of the pulse. In addition, an extended cavity Ti:Sa laser, an acousto-optical modulated (AOM) cavity dumped Ti:Sa laser, and even an amplified Ti:Sa laser can also be used as the light source. Typically these lasers are used because higher energy per pulse can be obtained than a standard Ti:Sa oscillator using these other variations. Newer ultrafast laser sources such as Ytterbium/Erbium doped broadband fiber lasers and Ytterbium:KGW solid-state femtosecond lasers, either femtosecond or picosecond pulsewidth, can also be used as the laser source in this invention.

Raman spectra of cells can be obtained with a single broadband coherent laser source through optical pulse shaping. A broadband coherent ultrafast laser 602 is used as the excitation light source and subsequently the amplitude, the phase and/or the polarization of the pulse (any or a combination), pulse width (compression and spreading) are pulse shaped. Raman spectroscopy of biological samples typically covers the “Raman fingerprint” region ranging from 0 to 1800 cm⁻¹. In microscopy, this range can be extended to above 3000 cm⁻¹ to reach to some higher vibrational frequencies, such as the CH₂ vibrational stretch of lipids at 2840 cm⁻¹. In order to cover such a wide frequency range, a broad optical spectrum is required. For example, for Ti:Sa femtosecond lasers with center wavelength at 800 nm, a broad spectrum from 700 nm to 900 nm is needed to cover Raman frequencies above 3000 cm⁻¹. Such a broad optical spectrum is typically not available from a femtosecond or picosecond laser source. Therefore, a photonic crystal fiber 604 (such as SCG-800 of Crystal fiber) can be used to generate a super-continuum spectrum from ultrafast pulses through nonlinear optical processes. As an alternative, a super-continuum fiber laser source (such as the SC500-FC of Fianium) can also be used directly as the broadband light source. The photonic crystal fiber is not a necessity in all embodiments of the design. For example, if only the Raman fingerprint region from 0 to 1800 cm⁻¹ is needed to be covered, a careful designed Ti:Sa oscillator can provide enough bandwidth ranging from 750 to 850 nm without other external elements.

Besides broadening an optical spectrum, the photonic crystal fiber 604 can also be used to tailor the optical spectrum to enhance the intensities of the probe wavelengths, as shown in FIG. 7B. The higher intensity probe allows more efficient generation of the CARS signals even if the number of vibrational oscillators in the sample is relatively low.

The broadband laser source is subsequently sent to an optical pulse shaper for intensity, phase and/or polarization shaping. In one embodiment, the optical pulse shaper is based on the standard 4-f pulse shaper geometry with a pair of dispersive grating 802, 816 (or dispersive prisms) combining with curved mirrors/lenses 804, 814 to spatially distribute the optical spectrum at the conjugate plane of the setup. A spatial light modulator (SLM) 806, such as SLM-128 of CRI, is used to modulate the phases and polarizations of each individual frequency across the optical spectrum. The SLM is not limited to use liquid-crystal based technology and other technology such as acousto-optic spatial light modulation can also be used to implement the optical pulse shaper. The amplitude shaping can also be implemented by inserting an opaque mask at the conjugate plane of the pulse shaper to block out some unwanted optical frequencies.

In one embodiment of the implementation of the multiplex CARS for cell sorting, the full CARS spectrum covering the whole Raman fingerprint region is obtained. In this case, the pulse shaper is used to compensate for high order material dispersion acquired by the pulse when the pulse propagates through the optical setup to generate a transform limited pulse at the sample. This allows all optical frequencies arrive at the sample at the same time in order to excite all vibrational frequencies through different frequency combinations across the whole optical spectrum of the pulse. However, a potential drawback of this approach is that the high optical intensity of the broadband source with the full optical spectrum incident on the cells could cause optical damage to the cells.

In such scenario, an alternative scanning approach can be used. The pulse shaper can be configured to allow only two (or a few) discreet frequencies to transmit through the SLM at a time. Since an SLM operates through either electro-optical or electroacousto effects and no mechanical movements are involved, rapid frequencies scanning is possible through sweeping the pump frequencies against the Stokes frequencies across the whole optical spectrum. Since more optical intensity can be concentrated at just these two frequencies to enhance the CARS signal, cell damage can be greatly reduced.

In addition, sometimes only a small Raman region or only several specific Raman frequencies are needed, because it has been determined that this small range of Raman spectrum will be adequate to differentiate cells. A small portion of the optical frequencies or these specific frequencies can also be “cut out” through intensity modulation by the pulse shaper to avoid putting in excessive optical energy into the cells.

The shaped optical pulse is now ready to send to the microfluidic device 500 to generate the CARS spectrum of the cell for cell sorting. A chromatic aberration corrected microscope objective is used to focus the pulses to the cells. The generated anti-Stokes frequencies are then collected either in the forward or epi direction for analysis.

In the embodiment of FIG. 8, a new approach is used to extract Raman scattering (as shown on the left of FIG. 7B) from CARS input pulses. An optical time-delay is inserted at the probe wavelengths 824, via optical delay device 810. This optical delay is realized by inserting an additional optical path to the probe wavelengths, for example using four mirrors 810. The non-resonance background is an instantaneous process and it only occurs when the three wavelengths arrive at the sample exactly at the same time. On the other hand, the CARS process requires only the pump and Stokes wavelengths 822 arrive at the sample at the same time, because the energy difference is actually stored in the vibrational motions of the molecules. The probe photon 824 can come to the sample a little later in time to retrieve this energy to generate the CARS signal 528. This approach eliminates the optics before the spectrometer, and only a one-dimensional spectrometer is required to measure the CARS spectrum.

The generated CARS signal 528 is finally sent to a detector 530 for data acquisition. Depending on the scheme to remove the non-resonance background, a one-dimensional spectrometer, a two-dimensional spectrometer, or a signal element detector, such as a photo-multiplier tube, can be used to acquire the data. In the embodiment of FIG. 8, a one-dimensional spectrometer is used. A two-dimensional spectrometer will be required if a 90-degree phase shift is added to the probe wavelength and subsequently mixed through two perpendicular polarizations (as in FIG. 7D). In the case where the SLM is configured to scan the vibrational frequencies across the desired spectrum, a single-element detector can be used since only one CARS frequency is detected at any particular time. The detector elements are not limited to a particular type of detector. For example, two-dimensional charged-couple detectors (CCDs), one-dimensional photo-multiplier tube (PMT) arrays, Silicon photo-detector arrays, single element photo-multiplier tubes, semiconductor photodiodes, and avalanche photodiodes can all be used for signal detections.

In the present invention, a galvo mirror 818 is used to dynamically guide pulses 526. This is useful for two reasons. First, if a longer acquisition is desired, the pulses can follow the target cell as it travels along the microfluidic device. Second, the pulses may be scanned across the width of the cell. Or, both may be accomplished at once.

Those skilled in the art will appreciate that the elements can be mixed and matched in a variety of ways, and that other elements not specifically shown and described could perform these functions. It will be appreciated by one skilled in the art that there are many possible variations on these designs that fall within the scope of the present invention.

What is claimed is:

1. A microfluidic cell sorter comprising:
a microfluidic structure (500) including a sample input channel (110) leading into an observation region (214), two buffer input channels (118) configured to hydrodynamically focus a target cell (208) in the input sample within the observation region, at least two output channels (114), and apparatus (536) for directing the target cell into a selected output channel based upon a cell sorting control signal (616);
a coherent anti-Stokes Raman scattering (CARS) pulse source (524) for generating CARS pulses;
apparatus configured to direct the CARS pulses into the target cell within the observation region;
a detector (530) configured to detect CARS illumination scattered from the target cell and to generate a spectrum signal based upon the detected illumination; and
a processor (614) configured to identify the target cell based upon the spectrum signal and further configured to generate the cell sorting control signal based upon the target identity.

2. The cell sorter of claim 1 wherein the apparatus for directing the target cell includes apparatus for applying back-pressure to an output channel that is not selected.

3. The cell sorter of claim 1 wherein the CARS pulse source includes a broadband laser source (602) configured to provide pump, probe and Stokes frequencies, and a pulse shaper (604, 606).

4. The cell sorter of claim 3 wherein the broadband laser source includes a femtosecond laser and a photonic crystal fiber (604) to broaden the pulse.

5. The cell sorter of claim 4 wherein the photonic crystal fiber further amplifies the intensity of the probe frequency.

6. The cell sorter of claim 3 wherein the broadband laser source comprises a fiber laser.

7. The cell sorter of claim 3, wherein the pulse shaper includes apparatus configured to delay (810) the probe wavelength.

8. The cell sorter of claim 1, wherein the microfluidic structure comprises a silicon structure (306) sandwiched between two glass slides (310).

9. The cell sorter of claim 8 wherein the silicon structure is anodically bonded to the slides and wherein the slides comprise polished pyrex.

10. A microfluidic cell flow cytometer (500) for examining cells in an input sample comprising:
a microfluidic structure including a sample input channel (110) leading into an observation region (214) and two buffer input channels (118) configured to hydrodynamically focus a continuous stream of cells (204, 208) in a single file line within the observation region;
a coherent anti-Stokes Raman scattering (CARS) pulse source (524) for generating CARS pulses;
apparatus (520) for detecting the presence of each of a series of target cells as they enter the observation region and for triggering the CARS pulse source accordingly;
apparatus (816, 818) configured to direct the CARS pulses into the target cells within the observation region;
a detector (530) configured to detect CARS illumination scattered from each target cell and to generate a spectrum signal based upon the detected illumination; and
a processor (614) configured to analyze each target cell based upon its associated spectrum signal.

11. The flow cytometer of claim 10 configured to analyze on the order of 100 cells per second.

12. The flow cytometer of claim 10 wherein the CARS pulse source includes a broadband laser source (602) configured to provide pump, probe and Stokes frequencies, and a pulse shaper (604, 606).

13. The flow cytometer of claim 12 wherein the broadband laser source includes a femtosecond laser and a photonic crystal fiber to broaden the pulse.

14. The flow cytometer of claim 13 wherein the photonic crystal fiber further amplifies the intensity of the probe frequency.

15. The flow cytometer of claim 12 wherein the broadband laser source comprises a fiber laser.

16. The flow cytometer of claim 12, wherein the pulse shaper delays (810) the probe wavelength.

17. The flow cytometer of claim 12 wherein the pulse shaper includes a spatial light modulator (806) configured to sweep the pump frequencies against the Stokes frequencies across an optical spectrum.

18. The flow cytometer of claim 12 wherein the pulse shaper is configured to apply intensity modulation to cut out selected optical frequency ranges.

19. The flow cytometer of claim 12 wherein the pulse shaper includes a galvo mirror (816) configured to dynamically guide pulses to follow the target cell as it travels through the observation region.
